(19) United States
(12) Patent Application Publication
(10) Pub. No.: US 2007/0292493 A1
(43) Pub. Date: Dec. 20, 2007

(54) Pharmaceutical composition and method for the transdermal delivery of calcium

(76) Inventor: Barbara T. Brierre, Lafayette, LA (US)

Correspondence Address: PERRET DOISE;A PROFESSIONAL LAW CORPORATION, P.O. DRAWER 3408, LAFAYETTE, LA, 70502-3408, US

(21) Appl. No.: 11/453,833

(22) Filed: Jun. 15, 2006

(51) Int. Cl. A61K 31/714; A61K 31/7048; A61K 31/57; A61K 31/59; A61K 31/525; A61K 31/56; A61K 31/4415; A61K 31/455; A61K 31/4188; A61K 31/405; A61K 31/51; A61K 31/355; A61K 31/045

(52) U.S. Cl. 424/449; 514/52; 514/171; 514/167; 514/251; 514/276; 514/350; 514/356; 514/456; 514/458; 514/27; 514/393; 514/725; 514/729; 514/419; 514/753

(57) ABSTRACT

The present invention relates to a method and transdermal pharmaceutical composition for preventing or reducing the likelihood of calcium deficiency or imbalances caused by calcium deficiency. The transdermal pharmaceutical composition includes a therapeutically effective amount of a pharmaceutically acceptable salt of calcium and a pharmaceutically acceptable carrier constituting a pluronic lecithin organogel. In addition to calcium, the transdermal pharmaceutical composition may also contain a therapeutically effective amount of: (1) a pharmaceutically acceptably salt of other minerals such as magnesium, zinc, selenium, manganese, or chromium; (2) a vitamin such as vitamin A, vitamin D, vitamin C, vitamin E or B-complex vitamins, choline, lecithin, inositol, PABA, biotin, or bioflavomoids; (3) a carotenoid such as lycopene or lutein; (4) a hormone such as dehydroepiandrosterone, progesterone, pregnenolone, or melatonin; (5) an amino acid such as arginine, glutamine, lysine, phenylalanine, tyrosine, GABA, tryptophan, carnitine, or acetyl-l-carnitine; (6) a fatty acid such as a fish oil or flax seed oil; (7) a vita-nutrient such as coenzyme Q10; (8) a cartilage building nutrient such as glucosamine, chondroitin, or MSM, (9) a herb such as ginkgo biloba, echinacea, 5-HTP, St. John's wort, or saw palmetto; or (9) any combination thereof. The transdermal pharmaceutical composition may be topically administered to a human to prevent or reduce the likelihood of calcium deficiency or imbalances caused by calcium deficiency such as hypertension, high cholesterol, colon and rectal cancer, osteomalacia, rickets, osteoporosis, cardiovascular disease, preeclampsia, tooth decay, and premenstrual syndrome.

### FIELD OF THE INVENTION

The present invention relates to a pharmaceutical composition for the transdermal delivery of calcium and to a method of topically administering the pharmaceutical composition to prevent or reduce the likelihood of calcium deficiency and imbalances caused by calcium deficiency.

### BACKGROUND OF THE INVENTION

Calcium is an essential mineral. The adult skeleton contains about 1,200 grams of calcium, 99% of which is stored in the bones and teeth. The remaining 1% of calcium (about 10-12 grams) circulates within the body in a soluble form. The role of calcium in maintaining strong bones and teeth is well known. Lesser known is the vital role soluble calcium plays in neuromuscular and cardiovascular function, in coagulation, as an intracellular second messenger for cell surface hormone action, and in gene transcription, cellular growth, and metabolism.

A synergistic relationship exists between calcium and magnesium. When the amount of magnesium in the bloodstream falls, the kidneys readjust the balance by holding onto less calcium. When magnesium concentration rises, the kidneys excrete less calcium. Therefore, the more magnesium a person ingests, the more calcium is kept in the person's body.

The recommended daily intake (RDI) of calcium is 1,000 mg for everyone over the age of four, except for pregnant and lactating women, for whom the RDI is 1,300 mg.

The primary dietary source of calcium is dairy products, such as milk, cheese and yogurt. Plants foods, such as tofu, kale, spinach, turnip greens, and other green leafy vegetables are also good sources.

Dietary sources of calcium are primarily responsible for maintaining circulating blood calcium. When diet is insufficient, the body will draw calcium out of the bones. Over time, this may lead to osteoporosis. It is estimated that 15 to 20 million Americans suffer from this disorder. Women over the age of 50 are particularly at risk.

Many disorders besides osteoporosis are attributable to calcium deficiency. Lack of sufficient calcium in children causes rickets, which in turn may cause bone deformity and growth retardation. Calcium deficiency in adults may result not only in osteomalacia or the softening of the bone. Extremely low blood levels of calcium may result in muscle spasms and leg cramps. Low calcium intake also contributes to high blood pressure, preeclampsia, and colon and rectal cancer. See, Weaver, Connie M., Calcium Requirements of Physically Active People, Am J Clinical Nutrition, 2000; 72:579S-584S; See also, See, Power, Michael L. et al, The Role of Calcium in Health and Disease, Am J Obstet Gynecol, 1999; 181:1560-1569. Calcium supplementation has also been found to reduce the symptoms of premenstrual syndrome. See, Power, Michael L. et al, The Role of Calcium in Health and Disease, Am J Obstet Gynecol, 1999; 181:1560-1569.

The body's absorption of calcium depends on calcium ionization in the intestines. Calcium ionization is a major problem with calcium carbonate, the most widely used calcium supplement. For calcium carbonate to be absorbed, it must first be solubilized and ionized by stomach acid. A study of post-menopausal women showed that about 40% of those studied were severely deficient in stomach acid. Patients with insufficient stomach acid output can only absorb about 4% of a calcium carbonate by oral dose whereas a person with normal stomach acid can absorb about 22%. See, Murray, Michael T., Encyclopedia of Nutritional Supplements, 1996, pgs. 149-158.

The effectiveness of calcium absorption from oral supplements is also dependent on other factors. For example, absorption may be affected by pH and other components of food such as oxalates and phytates. Calcium supplements are best taken in multiple doses rather than as a single dose to maximize absorption efficiency. As the number of daily supplement doses increases, subject compliance generally decreases. It is also important that calcium carbonate oral supplementation is taken with meals to optimize calcium bio-availability.

For these reasons, the oral ingestion of a calcium supplement is problematic. There exists a need for a novel pathway for calcium supplementation that avoids the problems associated with ingestion and promotes the maximum absorption of calcium in the body with the minimum of inconvenience.

### SUMMARY OF THE INVENTION

It is an object of the present invention to provide a delivery mechanism for calcium that alleviates the disadvantages associated with the oral administration of calcium supplements.

This object is achieved by the present invention that provides a pharmaceutical composition for the transdermal delivery of calcium that prevents or reduces the likelihood of calcium deficiency or imbalances caused by calcium deficiency in a human or other animal.

The transdermal pharmaceutical composition may contain a therapeutically effective amount of a pharmaceutically acceptable salt of calcium and a pharmaceutically acceptable carrier. The composition may be rubbed onto the outer layer of skin of a human or other animal to achieve the transdermal delivery of the therapeutically effective amount of the pharmaceutically acceptable salt of calcium.

The transdermal pharmaceutical composition may contain a therapeutically effective amount of calcium in the range of 10 mg to 150 mg per dosage. The therapeutically effective amount of calcium in the composition may preferably be about 40 mg per dosage. The pharmaceutically acceptable salt of calcium used in the composition may be calcium pantothenate, calcium citrate, calcium carbonate, calcium gluconate, or calcium lactate.

The pharmaceutically acceptable carrier may be a pluronic lecithin organogel. The pluronic lecithin organogel may comprise a mixture of a soy lecithin/isopropyl palmitate and a pluronic organogel.

In addition to calcium, the transdermal pharmaceutical composition may also include other compounds such as a therapeutically effective amount of other minerals, vitamins, carotenoids, hormones, amino acids, fatty acids, vita-nutrients, cartilage building nutrients, herbs, or any combination thereof.

The transdermal pharmaceutical composition may include a therapeutically effective amount of a mineral (other than calcium) such as a pharmaceutically acceptable salt of magnesium, zinc, selenium, manganese, chromium, or any combination thereof.

The transdermal pharmaceutical composition may include a therapeutically effective amount of a vitamin such as vitamin A, vitamin D, vitamin C, vitamin E, vitamin B₆, vitamin B₁₂, vitamin B₃, vitamin B₅, vitamin B₂, vitamin B₁, folic acid, choline, lecithin, inositol, para-aminobenzoic acid (“PABA”), vitamin H or B₇ and C₁₀H₁₆N₂O₃S (“biotin”), bioflavonoids, or any combination thereof.

The transdermal pharmaceutical composition may include a therapeutically effective amount of a carotenoid such as lycopene, lutein, or any combination thereof.

The transdermal pharmaceutical composition may include a therapeutically effective amount of a hormone such as dehydroepiandrosterone (“DHEA”), progesterone, pregnenolone, melatonin, or any combination thereof.

The transdermal pharmaceutical composition may include a therapeutically effective amount of an amino acid such as arginine, glutamine, lysine, phenylalanine, tyrosine, gamma-aminobutyric acid (“GABA”), tryptophan, carnitine, acetyl-l-carnitine, or any combination thereof.

The transdermal pharmaceutical composition may include a therapeutically effective amount of a fatty acid such as fish oil, flax seed oil, or a combination thereof.

The transdermal pharmaceutical composition may include a therapeutically effective amount of a vita-nutrient such as coenzyme Q10.

The transdermal pharmaceutical composition may include a therapeutically effective amount of a cartilage building nutrient such as glucosamine, chondroitin, methylsulfonylmethane (“MSM”), or any combination thereof.

The transdermal pharmaceutical composition may include a therapeutically effective amount of a herb such as ginkgo biloba, echinacea, 5-hydroxytryptophan (“5-HTP”), St. John's wort, saw palmetto, or any combination thereof.

The transdermal pharmaceutical composition may be topically administered in the appropriate dosage to prevent or reduce the likelihood of calcium deficiency or imbalances caused by calcium deficiency. Such imbalances include hypertension, high cholesterol, colon and rectal cancer, osteomalacia, rickets, osteoporosis, cardiovascular disease, preeclampsia, tooth decay, and premenstrual syndrome.

The transdermal pharmaceutical composition of the present invention is important because it bypasses the gastrointestinal tract thereby eliminating the side effects associated with oral ingestion of calcium supplements and the difficulties of intestinal absorption. The transdermal delivery of calcium achieves more effective and more consistent calcium supplementation.

Transdermal delivery of nutrients (e.g., calcium, magnesium. etc.) and hormones provide an 80-90% absorption rate directly to the blood stream and then from the blood stream to the tissue layers of the body where the are most effective. By contrast, the oral delivery of nutrients and hormones provide only 20-30% absorption rate to the tissue layer. Most of the nutrients and hormones in oral supplements are lost during passage through the gastrointestinal tract.

### DETAILED DESCRIPTION OF PREFERRED EMBODIMENTS OF INVENTION

The present invention is a transdermal pharmaceutical composition for preventing or reducing the likelihood of calcium deficiency and/or imbalances caused by calcium deficiency. The transdermal pharmaceutical composition may contain a therapeutically effective amount of a pharmaceutically acceptable salt of calcium and a pharmaceutically acceptable carrier.

Examples of pharmaceutically acceptable salts of calcium that may be used in the transdermal pharmaceutical composition include calcium carbonate, calcium pantothenate, calcium citrate, calcium gluconate, or calcium lactate. Calcium pantothenate is preferred.

The therapeutically effective amount of the pharmaceutically acceptable salt of calcium (e.g., calcium pantothenate) may be in the range of 10 mg to 150 mg per dosage and more particularly may be about 40 mg per dosage.

The transdermal pharmaceutical composition of the present invention may contain a therapeutically effective amount of a pharmaceutically acceptable salt of calcium, a therapeutically effective amount of other minerals, vitamins, carotenoids, hormones, amino acids, fatty acids, vita-nutrients, cartilage building nutrients, and/or herbs, and a pharmaceutically acceptable carrier.

Examples of other minerals that may be used in combination with calcium in the transdermal pharmaceutical composition include a therapeutically effective amount of magnesium, zinc, selenium, manganese, and/or chromium.

Magnesium is an essential mineral. It is the fourth most abundant cation in the human body and is present in more than 300 enzymatic systems, including adenosine triphosphate (ATP) metabolism, activation of creatine kinase, adenylate cyclase, and sodium potassium-ATPase. Magnesium functions physiologically in the body to control nerve action, heart activity, neuromuscular transmission, muscular contraction, vascular tone, blood pressure, and peripheral blood flow. Magnesium regulates the entry and release of calcium from cells which is determinative of muscular activity. The importance of magnesium to maintaining health and well-being cannot be overstated.

Magnesium is known to prevent or reduce the likelihood of numerous imbalances including mitral valve prolapse, dysautonomia, diabetes, cardiovascular diseases, high cholesterol, premenstrual syndrome, migraines, preeclampsia, asthma, high blood pressure, osteoporosis, muscle cramping, irritable bowel syndrome, fibromyalgia and chronic fatigue, kidney stones, and constipation.

Pharmaceutically acceptable salts of magnesium that may be used in the transdermal pharmaceutical composition include magnesium oxide, magnesium carbonate, magnesium chloride, magnesium sulfate, magnesium phosphate, magnesium bicarbonate, magnesium glycinate, magnesium aspartate, magnesium glutamate, magnesium adipate, magnesium citrate, magnesium orotate, magnesium taurate, and magnesium lysinate. Magnesium chloride is preferred.

The therapeutically effective amount of the pharmaceutically acceptable salt of magnesium (e.g., magnesium chloride) may be in the range of 4.0 mg to 100 mg per dosage and more particularly may be about 7.44 mg per dosage.

Zinc is a co-factor in hormonal metabolism, aids in the immune system, and helps build the collagen matrix of cartilage and bone.

Pharmaceutically acceptable salts of zinc that may be used in the transdermal pharmaceutical composition include zinc chloride. The therapeutically effective amount of the pharmaceutically acceptable salt of zinc (e.g., zinc chloride) may be in the range of 1 mg to 30 mg per dosage and more preferably may be about 12 mg per dosage.

Selenium's most important biological function relates to its role as an antioxidant and anticancer mineral. Selenium is an activating component of the enzyme glutathione peroxidase, which protects human body cells from damage. Selenium has also been shown to prevent heart disease.

The therapeutically effective amount of selenium that may be used in the transdermal pharmaceutical composition may be in the range of 20 mcg to 100 mcg per dosage and more preferably is about 20 mcg per dosage.

Manganese is an essential trace nutrient. It is involved in various enzyme systems that facilitate processes throughout the body, including protein, fat, and carbohydrate metabolism. Manganese is required for normal growth and development and for the repair of bones and connective tissue. It also plays a role in maintaining the proper function of nerves.

The therapeutically effective amount of manganese that may be used in the transdermal pharmaceutical composition may be in the range of 0.2 mg to 3,5 mg per dosage and more preferably is about 0.3 mg per dosage.

Chromium is responsible to activating enzymes involved in the metabolism of glucose and the synthesis of proteins. It is the major mineral responsible for insulin production. Therefore, lack of chromium interferes with the maintenance of healthy blood sugar levels. Evidence now suggests that chromium deficiency may cause many disorders of the glucose metabolism such as diabetes and hypoglycemia. It has also been implicated as a cause of high cholesterol.

The therapeutically effective amount of chromium that may be used in the transdermal pharmaceutical composition may be in the range of 20 mcg to 100 mcg per dosage and more preferably is about 20 mcg per dosage.

Examples of vitamins that may be used in combination with calcium in the transdermal pharmaceutical composition include a therapeutically effective amount of vitamin A, vitamin D, vitamin C, vitamin E, vitamin B₆, vitamin B₁₂, vitamin B₃, vitamin B₅, vitamin B₂, vitamin B₁, folic acid, choline, lecithin, inositol, PABA, biotin, and/or bioflavonoids.

Vitamin A (retinol) plays an important role in the immune system function, it helps protect the body from cardiovascular disease and cancer, it is required for the growth and maintenance of the skin and it is critical for the proper function of the eye. The vitamin A used may be vitamin A palmitate or vitamin A acetate. Vitamin A palmitate is preferred.

The therapeutically effective amount of vitamin A (e.g. vitamin A palmitate) may preferably be in the range of 70 IU to 500 IU per dosage and more preferably about 74.4 IU per dosage.

Vitamin D plays an important role in mineral absorption and bone mineralization and is extremely important in the maintenance of bone density. When combined with calcium, vitamin D has been found to possess anti-cancer properties. It has also been found to play a role in the treatment of immunological disorders such as multiple sclerosis and psoriasis. The vitamin D used may be cholecalciferol (D₃) calciferol (D₁), or ergocalciferol (D₂). Cholecalciferol (D₃) is preferred.

The therapeutically effective amount of vitamin D (e.g. cholecalciferol) may preferably be in the range of 7.0 IU to 1000 IU per dosage and more preferably about 7.44 IU per dosage.

Vitamin C (ascorbic acid) is an antioxidant that prevents free-radical damage often associated with aging and degenerative and age-related diseases, including cancer and cardiovascular disorders. Vitamin C plays a vital role in the immune system where it help increase resistance to a range of diseases including infections and cancer. Vitamin C is also known to help in the body's ability to handle all types of physical and mental stress. Vitamin C is essential for growth and repair of tissues in all parts of the body. It is needed for the formation of collagen, bones, and cartilage. It is well known that a deficiency in vitamin C leads to scurvy.

The therapeutically effective amount of vitamin C may be in the range of 3.0 mg to 200 mg per dosage and more preferably about 50 mg per dosage.

Vitamin E is a powerful antioxidant that plays a role in the prevention of age-related degenerative diseases such as cancer and cardiovascular disease. Vitamin E is several compounds including alpha-, beta-, delta-, and gamma-tocopherol; and alpha-, beta-, delta-, and gamma-tocotrienol. Most vitamin E supplements contain alpha-tocopherol. Vitamin E is known to reduce cholesterol, reduce inflammation, protect the nervous system, and studies have suggested that it may prevent dementia and Alzheimer's disease. Vitamin E is also known to play a role in the healing wounds and in the reduction of scar formation. It enhances the immune system.

The therapeutically effective amount of vitamin E may be in the range of 10 IU to 120 IU per dosage and more preferably about 20 IU per dosage.

Vitamin B₆ (pyridoxine) functions to increase the amount of magnesium that can enter cells and thus provides a synergistic and beneficial effect when combined with magnesium. Vitamin B₆ also facilitates the production of progesterone and reduces inflammatory reactions in connective tissue and collagen repair.

The therapeutically effective amount of vitamin B₆ may preferably be in the range of 2.0 mg to 20 mg per dosage and more preferably about 17.8 mg per dosage.

Vitamin B₁₂ (cobalamin, cyanocobalamin) assists in the proper absorption of other vitamins. Both vitamin B₆ and vitamin B₁₂ promote brain function, transfer food into energy within cells, and neutralize homocysteine which is a toxic by-product of protein metabolism and a risk factor for heart disease.

Stomach absorption of vitamin B₁₂ by oral dosing is problematic as there is minimal absorption of B₁₂ in the stomach due to the body's natural production of intrinsic factors. Thus, B₁₂ is normally delivered by intramuscular injection. The present invention avoids the disadvantages associated with oral delivery and/or intramuscular injection by providing for the transdermal delivery of vitamin B₁₂.

The therapeutically effective amount of vitamin B₁₂ may preferably be in the range of 10 mcg to 5000 mcg per dosage and more preferably about 420 mcg per dosage.

Vitamin B₃ is known to treat pellagra, correct niacin deficiency, reduce cholesterol and triglycerides in blood, dilate blood vessels if taken in doses larger than 75 mg, and treat vertigo (dizziness) and ringing in ears. Vitamin B₃ may also reduce the risk of heart attacks, may reduce depression, may reduce migraine headaches, and potentially improves poor digestion.

The therapeutically effective amount of vitamin B₃ may preferably be in the range of 10 mg to 300 mg per dosage and more preferably about 100 mg per dosage.

Vitamin B₅ (pantothenic acid) promotes normal growth and development, aids release of energy from foods, and helps synthesize numerous body materials. Vitamin B₅ may also stimulate wound healing, may alleviate stress, and may reduce fatigue.

The therapeutically effective amount of vitamin B₅ may preferably be in the range of 10 mg to 200 mg per dosage and more preferably about 60 mg per dosage.

Vitamin B₂ (riboflavin) aids the release of energy from food; maintains healthy mucous membranes lining the respiratory, digestive, circulating, and excretory tracts when used in conjunction with vitamin A; preserves integrity of the nervous system, skin, and eyes; promotes normal growth and development; activates vitamin B₆; and is essential for the conversion of tryptophan to niacin. Vitamin B₂ may also increase body growth during normal developmental stages and is a possible treatment for chellitis.

The therapeutically effective amount of vitamin B₂ may preferably be in the range of 0.5 mg to 40 mg per dosage and more preferably about 15 mg per dosage.

Vitamin B₁ (thiamine) functions to keep mucous membranes healthy; maintain normal function of the nervous system, muscles, heart; aid in the treatment of herpes zoster; promote normal growth and development; treat beriberi; and replace deficiency caused by alcoholism, cirrhosis, overactive thyroid, infection, breast feeding, absorption diseases, pregnancy, prolonged diarrhea, and burns. Vitamin B₁ may also reduce depression, fatigue, motion sickness, and may improve appetite and mental alertness.

The therapeutically effective amount of vitamin B₁ may preferably be in the range of 5 mg to 100 mg per dosage and more preferably about 10 mg per dosage.

Folic acid (Vitamin B9) is needed for proper formation of red blood cells. A deficiency of folic acid may result in anemia. Studies have shown that folic acid, B₆, and B₁₂ work together to lower homocysteine levels, which is implicated in cardiovascular disease and stroke. Folic acid supplementation may reduce the risk of cardiovascular disease and stroke. It is also known to treat dysplasia. During pregnancy, folic acid supplementation is recommended to avoid anemia in the mother and birth defects in the fetus. Folic acid also supports healthy nervous system function and the body's immune system.

The therapeutically effective amount of folic acid may preferably be in the range of 0.03 mg to 0.8 mg per dosage and more preferably about 0.61 mg per dosage.

Choline is a member of the B-complex group. It is involved in the body's use of fats and cholesterol and alleviates or prevents the accumulation of abnormal quantities of fat in the liver. It is used in the transport and metabolism of fats. Choline is also used by the body to make acetylcholine, which is a neurotransmitter that permits the sending of messages from nerve fiber to nerve fiber. A deficiency of choline may lead to neurological disorders such as Huntington's chorea, Parkinson's disease, and Alzheimer's disease.

The therapeutically effective amount of choline may preferably be in the range of 20 mg to 1000 mg per dosage and more preferably about 50 mg per dosage.

Lecithin (phosphatidyl choline) is a natural source of choline. It is structural component of cell membranes. It has been used to treat the neurological disorder known as tardive dyskinesia. It has also been used to treat high levels of serum cholesterol.

The therapeutically effective amount of lecithin may preferably be in the range of 20 mg to 1000 mg per dosage and more preferably about 50 mg per dosage.

Inositol is also a B-complex vitamin. It is involved in the synthesis of phospholipids (essential to digestion and absorption of fats), the uptake of fatty acids by cells, and regulate the transport of material in and out of cells. Inositol has been found effective in treating depression, panic disorders, and obessive-compulsive disorder.

The therapeutically effective amount of inositol may preferably be in the range of 10 mg to 200 mg per dosage and more preferably about 50 mg per pump.

PABA is required for the body's formation of folic acid and for the metabolism of proteins.

The therapeutically effective amount of PABA may preferably be in the range of 600 mg to 1200 mg per dosage and more preferably about 1000 mg per dosage.

Biotin is not a true vitamin because it is made by intestinal bacteria. It is, however, an important coenzyme that is involved in numerous body processes. For example, biotin is necessary for the metabolism of carbohydrates, fats, and protein. Biotin deficiency results in seborrheic dermatitis and hair loss. It is also known to result in appetite loss, nausea, numbness, depression, and high blood cholesterol. Some evidence has demonstrated that biotin supplementation helps to prevent and treat nervous system disorders in person's undergoing long-term hemodialysis.

The therapeutically effective amount of biotin may preferably be in the range of 10 mcg to 300 mcg per dosage and more preferably about 100 mcg per dosage.

Bioflavonoids are a group of crystalline compounds found in plants such as quercetin, rutin, naringin, hesperidin, genistein, baicalin, pycnogenol, catechin, and bioflavonoid complex. They are antioxidants. Studies have also shown that bioflavonoids may play a role in lowering cholesterol and offering protection against cardiovascular disease. They also possess antiviral, anticancer, anti-inflammatory, and antihistamine activities.

The therapeutically effective amount of the bioflavonoid may preferably be in the range of 5 mg to 400 mg per dosage and more preferably about 100 mg per dosage.

Examples of carotenoids that may be used in combination with calcium in the transdermal pharmaceutical composition include a therapeutically effective amount of a beta-carotene such as lycopene and lutein.

Lycopene is known to reduce the risk of certain cancers.

The therapeutically effective amount of lycopene may preferably be in the range of 50 mcg to 500 mcg per dosage and more preferably about 420 mcg per dosage.

Lutein is known to help eye problems.

The therapeutically effective amount of lutein may preferably be in the range of 1 mg to 10 mg per dosage and more preferably about 2 mg per dosage.

Examples of hormones that may be used in combination with calcium in the transdermal pharmaceutical composition include a therapeutically effective amount of DHEA, progesterone, pregnenolone, and/or melatonin.

DHEA may reduce the likelihood or delay the onset of cancer, hardening of the arteries, lethal viral infections, lowered immunity, obesity, and diabetes. It has been suggested that DHEA may help treat the autoimmune disease lupus.

The therapeutically effective amount of DHEA may preferably be in the range of 2.5 mg to 10 mg per dosage and more preferably about 5 mg per dosage.

It has been found that when a therapeutically effective amount of progesterone is included in the transdermal pharmaceutical composition of the present invention the composition exhibits an enhanced ability to prevent imbalances associated with premenstrual syndrome as for example by preventing pain and cramping associated with premenstrual syndrome or menstruation.

The therapeutically effective amount of progesterone may preferably be in the range of 5 mg to 20 mg per dosage and more preferably about 20 mg per dosage.

Pregnenolone is a steroid hormone involved in the steriodogenesis of progesterone, mineralocorticoids, glucocorticoids, androgens, and estrogens. It is found in high concentrations in certain areas of the brain and is synthesized there. Pregnenoline may improve cognitive and memory function.

The therapeutically effective amount of pregnenolone may preferably be in the range of 2.0 to 30 mg per dosage and more preferably about 20 mg per dosage.

Melatonin is a hormone secreted by the pineal gland. It is found in every cell of every living organism. It is responsible for regulating biological rhythms. It has been used to induce sleep, reduce jet lag, and resolve confused body rhythms caused by shift work.

The therapeutically effective amount of melatonin may preferably be in the range of 0.5 mg to 20 mg per dosage and more preferably about 2 mg per dosage.

Examples of amino acids that may be used in combination with calcium in the transdermal pharmaceutical composition include a therapeutically effective amount of arginine, glutamine, lysine, phenylalanine, tyrosine, GABA, tryptophan, carnitine, and/or acetyl-l-carnitine.

Arginine plays an important role in cell division, the healing of wounds, removing ammonia from the body, immune function, and the release of hormones.

The therapeutically effective amount of arginine may preferably be in the range of 1 mg to 20 mg per dosage and more preferably about 2.1 mg per dosage.

Glutamine is a supplement used in weightlifting and bodybuilding as well as by those who suffer from muscular cramps or pain. It replenishes amino acid stores that have been depleted by exercise or everyday activities.

The therapeutically effective amount of glutamine may preferably be in the range of 200 mg to 500 mg per dosage and more preferably about 300 mg per dosage.

Lysine is an essential amino acid. A deficiency in lysine can result in a deficiency in niacin, which can cause pellagra. Lysine can also be used as a supplement to help against herpes.

The therapeutically effective amount of lysine may preferably be in the range of 100 mg to 200 mg per dosage and more preferably about 100 mg per dosage.

Phenylalanine is also an essential amino acid. It exists in two forms: D- and L-forms. It can be used to fight chronic pain and depression, including the mood swings caused by PMS. It may also increase energy and metal alertness.

The therapeutically effective amount of phenylalanine may preferably be in the range of 25 mg to 100 mg per dosage and more preferably about 50 mg per dosage.

Tyrosine is used by cells to synthesize proteins.

The therapeutically effective amount of tyrosine may preferably be in the range of 100 mg to 200 mg per dosage and more preferably about 150 mg per dosage.

GABA is an inhibitory neurotransmitter found in the nervous system. GABA supplementation aids in the treatment of anti-anxiety disorders and anti-convulsiveness.

The therapeutically effective amount of GABA may preferably be in the range of 50 mg to 400 mg per dosage and more preferably about 100 mg per dosage.

Tryptophan is an essential amino acid. It is a precursor for serotonin, melatonin, and niacin. Tryptophan deficiency has been implicated as a possible cause of schizophrenia and its supplementation has been indicated as an aid for schizophrenic patients. Tryptophan has been used as a sleep aid and to reduce chronic pain and impulsive, violent, manic, addictive, obsessive or compulsive disorders.

The therapeutically effective amount of tryptophan may preferably be in the range of 20 mg to 200 mg per dosage and more preferably about 100 mg per dosage.

Carnitine, also known as L-carnintine, is an amine derived from the amino acid lysine. It is responsible for the transport of fatty acids from the cytosol into a cell's mitochondria.

Acetyl-l-carnitine is an acetylated form of L-carnitine. It has been marketed as a life extension supplement. Other attributed uses of the compound include the treatment for depression, clearing of fatty deposits from veins and arteries.

The therapeutically effective amount of carnintine and/or acetyl-l-carnitine may preferably be in the range of 1 mg to 100 mg per dosage and more preferably about 2,1 mg per dosage.

Examples of fatty acids that may be used in combination with calcium in the transdermal pharmaceutical composition include a therapeutically effective amount of fish oil and/or flax seed oil.

Fish oil is derived from the tissues of oily fish. The oil contains Omega-3 fatty acids eicosapentaenoic acid (“EPA”) and docosahexaenoic acid (“DHA”). It is believed the oil helps regulate cholesterol because of the oil's anti-inflammatory properties.

The therapeutically effective amount of fish oil may preferably be in the range of 100 mg to 200 mg per dosage and more preferably about 150 mg per dosage.

Flax seed oil (also known as linseed oil) is derived from the dried ripe seeds of the flax plant. It is high in Omega-3 fatty acids, particularly alpha-linolenic acid. The benefits of Omega-3 fatty acids are discussed above.

The therapeutically effective amount of flax seed oil may preferably be in the range of 50 mg to 500 mg per dosage and more preferably about 100 mg per dosage.

Examples of vita-nutrients that may be used in combination with calcium in the transdermal pharmaceutical composition include a therapeutically effective amount of coenzyme Q10.

Coenzyme Q10 is also known as ubiquinone. It behaves like a vitamin because it serves as a catalyst in certain reactions, including the important reaction that produces ATP, a compound that yields energy needed by cells to function. Coenzyme Q10 is required for optimum health. It is an antioxidant. Research has shown that coenzyme Q10 supplementation aids in the reduction of angina. It may also protect the heart from damage due to heart attack. Studies suggest that it reduces the amount of tissue damage that occurs during open heart surgery. It may also be effective in the treatment of congestive heart failure. Evidence shows that coenzyme Q10 may decrease arrhythmia, help in the treatment of mitral valve prolapse, lower high blood presssure, and reduce bad cholesterol. A study suggests that coenzyme Q10 may aid patients with chronic fatigue and immune dysfunction.

The therapeutically effective amount of coenzyme Q10 may preferably be in the range of 9 mg to 40 mg per dosage and more preferably about 20 mg per dosage.

Examples of cartilage building nutrients that may be used in combination with calcium in the transdermal pharmaceutical composition include a therapeutically effective amount of glucosamine, chondroitin, and/or MSM.

Glucosamine is an amino sugar that is an important precursor in the biochemical synthesis of glycosylated proteins and lipids. It is used in the treatment of osteoarthritis.

The therapeutically effective amount of glucosamine may preferably be in the range of 50 mg to 300 mg per dosage and more preferably about 150 mg per dosage.

Chondroitin, namely in the form of chondroitin sulfate, is a structural component of cartilage. It is widely used as a dietary supplement for the treatment of osteoartritis, particularly when combined with glucosamine.

The therapeutically effective amount of chondroitin may preferably be in the range of 25 mg to 100 mg per dosage and more preferably about 50 mg per dosage.

MSM is an organic sulfur compound belonging to a class of chemicals called sulfones. It is the primary metabolite of DMSO (dimethyl sulfoxide) in humans, with which is shares certain properties. MSM is used as a dietary supplement to treat osteoarthritis. It is often used in combination with glucosamine and chondroitin. It has also been studied as a treatment for allergic rhinitis, interstitial cystitis, and snoring.

The therapeutically effective amount of MSM may preferably be in the range of 25 mg to 200 mg per dosage and more preferably about 50 mg per dosage.

Examples of herbs that may be used in combination with calcium in the transdermal pharmaceutical composition include a therapeutically effective amount of ginkgo biloba, echinacea, 5-HTP, St. John's wort, capsaicin, and/or saw palmetto.

Ginkgo biloba is an extract of the ginkgo tree that contains flavomoid glycosides. It has been used pharmaceutically as a memory enhancer and as an anti-vertigo agent. Ginkgo extracts has been shown to improve blood flow to tissues and organs, protect against oxidative cell damage from free radicals, and block the effects of PAF (platelet aggregation, blood clotting) that have been related to cardiovascular, renal, respiratory and central nervous system disorders.

The therapeutically effective amount of ginkgo biloba may preferably be in the range of 12 mg to 36 mg per dosage and more preferably about 16 mg per dosage.

Echinacea is derived from the family of flowering plants known as Asteraceae. Echinaceae rhizomes have been used for many years as an immune system booster to ward off infections, such as the common cold. Its health benefits have been attributable to the chemical called phenols contained with the compound.

The therapeutically effective amount of echinaceae may preferably be in the range of 76 mg to 104 mg per dosage and more preferably about 100 mg per dosage.

5-HTP is a precursor to the neurotransmitter serotonin and an intermediate in tryptophan metabolism. It has been marketed as a dietary supplement for use as an anti-depressant, appetite suppressant, and sleep aid. It is an amino acid.

The therapeutically effective amount of 5-HTP may preferably be in the range of 30 mg to 50 mg per dosage and more preferably about 40 mg per dosage.

St. John's wort is derived from the plant species Hypericum perforatum. It has been used for medicinal purposes for thousands of years. St. John's wort ha sbeen used as an anti-inflammatory, astrigent, and antiseptic. It has been used to treat depression and anxiety disorders.

The therapeutically effective amount of St. John's wort serine may preferably be in the range of 30 mg to 100 mg per dosage and more preferably about 30 mg per dosage.

Saw palmetto is an extract from the fruit of the small palm plant known as Serenoa repens. It has been used to treat urinary and genital problems, including benign prostatic hyperplasia.

The therapeutically effective amount of saw palmetto may preferably be in the range of 10 mg to 32 mg per dosage and more preferably about 11.42 mg per dosage.

As stated above, the transdermal pharmaceutical composition of the invention includes a pharmaceutically acceptable carrier for the active drug or supplement component. The pharmaceutically acceptable carrier preferably includes a pluronic lecithin organogel. The pluronic lecithin organogel may preferably be a mixture of soy lecithin/isopropyl palmitate syrup or solution and Pluronic F127 gel.

Pluronics (e.g., Pluronic F127 gel) are poloxamers. Poloxamers are co-polymers of polyoxyethylene and polyoxypropylene. Pluronics are commercially available from BASF Corporation. Pluronics form thermoreversible gels in concentrations ranging from 15% to 50%. This means they are liquids at cool (refrigerator) temperature, but are gels at room or body temperature. This characteristic is useful in pharmaceutical compounding because the Pluronics can be drawn into a syringe for accurate dose measurement when cold. When warmed to body temperature—as when applied to the skin—it thickens into a gel consistency that is odorless, colorless, and non-greasy. The thickening of the gel on the skin is rapid. After thickening, the gel penetrates the skin and leaves only a small amount of residue.

By combining Pluronic F127 gel (preferably Pluronic F127 20% gel) and a soy lecithin/isopropyl palmitate syrup or solution (thus resulting in what is known as a “PLO gel”), skin absorption characteristics are enhanced. To explain how skin absorption occurs, it is necessary to understand the composition of the skin.

The skin is composed of three major components: the epidermis, the dermis, and the underlying subdermal tissue. The epidermis, which provides the strongest protection against drug absorption, is composed of five different layers: stratum corneum, stratum lucidum, stratum granulosum, stratum spinosum, and stratum basale. Of these five layers, the stratum corneum is the most impermeable. It is made of flattened, cornified cells embedded in a lipid intercellular matrix.

PLO gels permeate the skin by two proposed mechanisms. The first mechanism proposes that the PLO gel with the active drug diffuses through the lipid intercellular matrix of the stratum corneum. The second mechanism proposes that the PLO gel provides a slight disorganization of the skin allowing permeation of the gel and the active drug through the stratum corneum. The lecithin component of the PLO gel (which is lipophilic) has the ability to act as an amphoteric surfactant and enables drugs to penetrate through the stratum corneum. When a water-soluble drug is added to a hydrophobic substance with the aid of a surfactant, both the drug and the hydrophobic medium can pass through the epidermis. Bioavailability ranges from 10% to 60%.

The transdermal pharmaceutical composition of the invention may be used in a method to prevent or reduce the likelihood of calcium deficiency and/or imbalances caused by or associated with calcium deficiency. These imbalances include hypertension, high cholesterol, colon and rectal cancers, osteomalacia, rickets, osteoporosis, cardiovascular disease, preeclampsia, tooth decay, and premenstrual syndrome.

The transdermal pharmaceutical composition of the invention should be applied to clean, hairless areas of the body such as the inside of the forearms, upper chest, and upper thigh. The PLO gel will form a deposit on the skin that provides sustained release of the active drug or supplement, e.g., pharmaceutically acceptable salts of calcium, pharmaceutically acceptable salts of other minerals (magnesium, zinc, selenium, manganese, and/or chromium), vitamins (vitamin A, vitamin D, vitamin B₆, vitamin B₁₂ vitamin B₃, vitamin B₅, vitamin B₂, vitamin B₁, folic acid, choline, lecithin, inositol, PABA, biotin, and/or bioflavonoids), carotenoids (lycopene and/or lutein), hormones (DHEA, progesterone, pregnenolone, and/or melatonin), amino acids (arginine, glutamine, lysine, phenylalanine, tyrosine, GABA, tryptophan, carnitine, and/or acetyl-l-carnitine), fatty acids (fish oil and/or flax seed oil), vita-nutrients (coenzyme Q10), cartilage building nutrients (glucosamine, chondroitin, and/or MSM), and/or herbs (ginkgo biloba, echinacea, 5-HTP, St. John's wort, and/or saw palmetto).

The transdermal pharmaceutical composition of the invention may be self-administered. For self-administration, the transdermal pharmaceutical composition may be placed in a dispenser (e.g., pump) that can be manipulated to provide the suitable dosage. The dosage is preferably 1 ml per day. If a pump is used to administer the composition, each manipulation of the pump should deposit 1 ml of the composition onto the skin.

Provided below are formulation examples which describe the preparation of the transdermal pharmaceutical composition of the invention and therapeutic examples which describe results obtained or expected from transdermal administration to human patients.

### Formulations

### EXAMPLE 1

### Calcium Composition

| Chemicals | Quantity |
|---|---|
| Deionized Water | 70.16% |
| Polyethylene-Polypropylene Glycol | 10.0% |
| Isopropyl Palmitate | 4.9% |
| Lecithin | 4.9% |
| Sodium Acryloyldimethyl, Taurate Copolymer, | 3.2% |
| Isohexadecane, and Polysorbate | |
| Calcium Pantothenate | 5.0% |
| Phenoxyethanol, Caprylyl Glycol, and Sorbic Acid | 1.5% |
| Potassium Sorbate | 0.15% |
| Sorbic Acid | 0.15% |
| Sodium Hydroxide | 0.04% |

The pharmaceutical composition contains a therapeutically effective amount of calcium pantothenate. This preferably is 37.2 mg/pump.

The preparation of the pharmaceutical composition with calcium pantothenate as the primary active ingredient is conducted in six separate phases: Liposome Oil Phase, Pluronic Phase, Oil Phase, Water Phase, Emulsifier Phase, and Final Mixing Phase.

Each of these phases is described below.

### I. The Liposome Oil Phase

In this phase, 2793.0 mg lecithin, 2793.0 mg isopropyl palmitate, and 85.05 mg sorbic acid are combined. Each ingredient is weighed in separate containers. The ingredients are combined and mixed with a drill mixer for at least 10 minutes in a pail. The pail is sealed with a lid and left to sit for at least 18 hours at room temperature.

### II. The Pluronic Phase

In this phase, 85.05 mg potassium sorbate, 22594.95 mg water, and 5670 mg poloxamer 407 are combined. Weigh each ingredient in separate containers and combine in a container. Mix with drill mixer for at least 1 minute. Seal with a lid and place in refrigerator for at least 18 hours.

### III. Oil Phase

In this phase, 850.5 mg optiphen plus is combined with the mixture formulated in Phase II (Pluronic Phase). Weigh ingredients in final mixing container and mix with drill mixer for at least 5 minutes.

### IV. Water Phase

In this phase, 17185.8 mg water and 2835 mg calcium pantothenate are combined. Weight water and calcium pantothenate separately and combine into a container. Mix with drill mixer for at least 5 minutes.

### V. Emulsifier Phase

In this phase, 1814.4 mg simugel 600 is used. Weigh ingredient in container and set it aside until ready to add.

### VI. Final Mixing Phase

Slowly add Phase II (Pluronic Gel) to Phase III (Oil Phase) while mixing with drill mixer. Mix for at least 2 minutes. Immediately add Phase IV (Water Phase) and mix for at least 10 minutes. Add Phase V (Emulsifier) and mix for at least 3 minutes. Set container aside until composition is at room temperature.

### EXAMPLE 2

### Calcium and Magnesium Composition

| Chemicals | Quantity |
|---|---|
| Deionized Water | 69.16% |
| Polyethylene-Polypropylene Glycol | 10.0% |
| Isopropyl Palmitate | 4.9% |
| Lecithin | 4.9% |
| Sodium Acryloyldimethyl, Taurate Copolymer, | 3.2% |
| Isohexadecane, and Polysorbate | |
| Calcium Pantothenate | 5.0% |
| Magnesium Chloride | 1.0% |
| Phenoxyethanol, Caprylyl Glycol, and Sorbic Acid | 1.5% |
| Potassium Sorbate | 0.15% |
| Sorbic Acid | 0.15% |
| Sodium Hydroxide | 0.04% |

The pharmaceutical composition contains a therapeutically effective amount of calcium pantothenate and magnesium chloride. This preferably is 37.2 mg/pump of calcium pantothenate and 7.44 mg/pump of magnesium chloride.

The preparation of the pharmaceutical composition with calcium pantothenate and magnesium chloride as the primary active ingredients is conducted in six separate phases: Liposome Oil Phase, Pluronic Phase, Oil Phase, Water Phase, Emulsifier Phase, and Final Mixing Phase. Each of these phases is described below.

### I. The Liposome Oil Phase

In this phase, 2793.0 mg lecithin, 2793.0 mg isopropyl palmitate, and 85.05 mg sorbic acid are combined. Each ingredient is weighed in separate containers. The ingredients are combined and mixed with a drill mixer for at least 10 minutes in a pail. The pail is sealed with a lid and left to sit for at least 18 hours at room temperature.

### II. The Pluronic Phase

In this phase, 85.05 mg potassium sorbate, 22594.95 mg water, and 5670 mg poloxamer 407 are combined. Weight each ingredient in separate containers and combine in a container. Mix with drill mixer for at least 1 minute. Seal with a lid and place in refrigerator for at least 18 hours.

### III. Oil Phase

In this phase, 850.5 mg optiphen plus is combined with the mixture formulated in Phase II (Pluronic Phase). Weigh ingredients in final mixing container and mix with drill mixer for at least 5 minutes.

### IV. Water Phase

In this phase, 16618.8 mg water, 2835 mg calcium pantothenate, and 567 mg magnesium chloride are combined. Weigh water, calcium pantothenate, and magnesium chloride separately and combine into a container. Mix with drill mixer for at least 5 minutes.

### V. Emulsifier Phase

In this phase, 1814.4 mg simugel 600 is used. Weigh ingredient in container and set it aside until ready to add.

### VI. Final Mixing Phase

Slowly add Phase II (Pluronic Gel) to Phase III (Oil Phase) while mixing with drill mixer. Mix for at least 2 minutes. Immediately add Phase IV (Water Phase) and mix for at least 10 minutes. Add Phase V (Emulsifier) and mix for at least 3 minutes. Set container aside until composition is at room temperature.

### EXAMPLE 3

### Calcium, Magnesium, Vitamin A, and Vitamin D Composition

| Chemicals | Quantity |
|---|---|
| Deionized Water | 69.06% |
| Polyethylene-Polypropylene Glycol | 10.0% |
| Isopropyl Palmitate | 4.9% |
| Lecithin | 4.9% |
| Sodium Acryloyldimethyl, Taurate Copolymer, | 3.2% |
| Isohexadecane, and Polysorbate | |
| Calcium Pantothenate | 5.0% |
| Magnesium Chloride | 1.0% |
| Phenoxyethanol, Caprylyl Glycol, and Sorbic Acid | 1.5% |
| Potassium Sorbate | 0.15% |
| Sorbic Acid | 0.15% |
| Vitamin A Palmitate and Cholecalciferol (D3) | 0.1% |
| Sodium Hydroxide | 0.04% |

The pharmaceutical composition contains a therapeutically effective amount of calcium pantothenate, magnesium chloride, vitamin D (cholecalciferol), and vitamin A palmitate. This preferably is 37.2 mg/pump of calcium pantothenate, 7.44 mg/pump of magnesium chloride, 7.44 IU/pump topical or 0.000019 mg/pump of vitamin D (cholecalciferol), and 74.4 IU/pump topical or 0.00019 mg/pump of vitamin A palmitate.

The preparation of the pharmaceutical composition with calcium pantothenate, magnesium chloride, vitamin D (cholecalciferol) and vitamin A palmitate as the primary active ingredients is conducted in six separate phases: Liposome Oil Phase, Pluronic Phase, Oil Phase, Water Phase, Emulsifier Phase, and Final Mixing Phase. Each of these phases is described below.

### I. The Liposome Oil Phase

In this phase, 2793.0 mg lecthin, 2793.0 mg isopropyl palmitate, and 85.05 mg sorbic acid are combined. Each ingredient is weighed in separate containers. The ingredients are combined and mixed with a drill mixer for at least 10 minutes in a pail. The pail is sealed with a lid and left to sit for at least 18 hours at room temperature.

### II. The Pluronic Phase

In this phase, 85.05 mg potassium sorbate, 22594.95 mg water, and 5670 mg poloxamer 407 are combined. Weight each ingredient in separate containers and combine in a container. Mix with drill mixer for at least 1 minute. Seal with a lid and place in refrigerator for at least 18 hours.

### III. Oil Phase

In this phase, 850.5 mg optiphen plus and 56.7 mg vitamin A palmitate and vitamin D (cholecalciferol) are combined with the mixture formulated in Phase II (Pluronic Phase). Weigh ingredients in final mixing container and mix with drill mixer for at least 5 minutes.

### IV. Water Phase

In this phase, 16556.4 mg water, 2835 mg calcium pantothenate, and 567 mg magnesium chloride are combined. Weigh water, calcium pantothenate, and magnesium chloride separately and combine into a container. Mix with drill mixer for at least 5 minutes.

### V. Emulsifier Phase

In this phase, 1814.4 mg simugel 600 is used. Weigh ingredient in container and set it aside until ready to add.

### VI. Final Mixing Phase

Slowly add Phase II (Pluronic Gel) to Phase III (Oil Phase) while mixing with drill mixer. Mix for at least 2 minutes. Immediately add Phase IV (Water Phase) and mix for at least 10 minutes. Add Phase V (Emulsifier) and mix for at least 3 minutes. Set container aside until composition is at room temperature.

While preferred embodiments of the present invention have been described, it is to be understood that the embodiments described are illustrative only and that the scope of the invention is to be defined solely by the appended claims when accorded a full range of equivalence, many variations and modifications naturally occurring to those skilled in the art from a perusal hereof.

What is claimed is:

1. A transdermal pharmaceutical composition for reducing the likelihood of calcium deficiency or imbalances caused by calcium deficiency in a human comprising:
a therapeutically effective amount of a pharmaceutically acceptable salt of calcium; and
a pharmaceutically acceptable carrier comprising a pluronic lecithin organogel;
wherein a dosage of said composition is rubbed onto an outer layer of skin of said human to achieve said transdermal delivery of said therapeutically effective amount of said pharmaceutically acceptable salt of calcium.

2. The transdermal pharmaceutical composition according to claim 1, wherein said therapeutically effective amount of calcium is in the range of 10 mg to 150 mg per dosage.

3. The transdermal pharmaceutical composition according to claim 2, wherein said therapeutically effective amount of calcium is 40 mg per dosage.

4. The transdermal pharmaceutical composition according to claim 1, wherein said pharmaceutically acceptable salt of calcium is calcium pantothenate.

5. The transdermal pharmaceutical composition according to claim 1, further comprising a therapeutically effective amount of a pharmaceutically acceptable salt of a mineral selected from the group consisting of magnesium, zinc, selenium, manganese, chromium, and any combination thereof.

6. The transdermal pharmaceutical composition according to claim 1, further comprising a therapeutically effective amount of a vitamin selected from the group consisting of vitamin A, vitamin D, vitamin C, vitamin E, vitamin B₆, vitamin B₁₂, vitamin B₃, vitamin B₅, vitamin B₂, vitamin B₁, folic acid, choline, lecithin, inositol, PABA, biotin, bioflavonoids, and any combination thereof.

7. The transdermal pharmaceutical composition according to claim 1, further comprising a therapeutically effective amount of a carotenoid selected from the group consisting of lycopene, lutein, and a combination thereof.

8. The transdermal pharmaceutical composition according to claim 1, further comprising a therapeutically effective amount of a hormone selected from the group consisting of dehydroepiandrosterone, progesterone, pregnenolone, melatonin, and any combination thereof.

9. The transdermal pharmaceutical composition according to claim 1, further comprising a therapeutically effective amount of an amino acid selected from the group consisting of arginine, glutamine, lysine, phenylalanine, tyrosine, GABA, tryptophan, carnitine, acetyl-l-carnitine, and any combination thereof.

10. The transdermal pharmaceutical composition according to claim 1, further comprising a therapeutically effective amount of a fatty acid selected from the group consisting of a fish oil, a flax seed oil, and any combination thereof.

11. The transdermal pharmaceutical composition according to claim 1, further comprising a therapeutically effective amount of coenzyme Q10.

12. The transdermal pharmaceutical composition according to claim 1, further comprising a therapeutically effective amount of a cartilage building nutrient selected from the group consisting of glucosamine, chondroitin, MSM, and any combination thereof.

13. The transdermal pharmaceutical composition according to claim 1, further comprising a therapeutically effective amount of a herb selected from the group consisting of ginkgo biloba, echinacea, 5-HTP, St. John's wort, saw palmetto, and any combination thereof.

14. The transdermal pharmaceutical composition according to claim 1, wherein said pluronic lecithin organogel comprises a mixture of a soy lecithin/isopropyl palmitate and a pluronic organogel.

15. The transdermal pharmaceutical composition according to claim 1, wherein said imbalances caused by calcium deficiency are selected from the group consisting of hypertension, high cholesterol, colon and rectal cancer, osteomalacia, rickets, osteoporosis, cardiovascular disease, preeclampsia, tooth decay, and premenstrual syndrome.

16. A transdermal pharmaceutical composition for reducing the likelihood of calcium deficiency or imbalances caused by calcium deficiency in a human comprising:
a therapeutically effective amount of calcium pantothenate;
a therapeutically effective amount of magnesium chloride; and
a pharmaceutically acceptable carrier comprising a pluronic lecithin organogel;
wherein a dosage of said composition is rubbed onto an outer layer of skin of said human to achieve said transdermal delivery of said therapeutically effective amount of said calcium pantothenate and said magnesium chloride.

17. The transdermal pharmaceutical composition according to claim 16, wherein said therapeutically effective amount of said calcium pantothenate is in the range of 10 mg to 150 mg per dosage and said therapeutically effective amount of said magnesium chloride is in the range of 4.0 to 100 mg per dosage.

18. The transdermal pharmaceutical composition according to claim 17, wherein said therapeutically effective amount of said calcium pantothenate is 40 mg per dosage and said therapeutically effective amount of said magnesium chloride is 7.44 mg per dosage.

19. The transdermal pharmaceutical composition according to claim 16, further comprising a therapeutically effective amount of a pharmaceutically acceptable salt of a mineral selected from the group consisting of zinc, selenium, manganese, chromium, and any combination thereof.

20. The transdermal pharmaceutical composition according to claim 16, further comprising a therapeutically effective amount of a vitamin selected from the group consisting of vitamin A, vitamin D, vitamin C, vitamin E, vitamin B₆, vitamin B₁₂, vitamin B₃, vitamin B₅, vitamin B₂, vitamin B₁, folic acid, choline, lecithin, inositol, PABA, biotin, bioflavonoids, and any combination thereof.

21. The transdermal pharmaceutical composition according to claim 16, further comprising a therapeutically effective amount of a carotenoid selected from the group consisting of lycopene, lutein, and a combination thereof.

22. The transdermal pharmaceutical composition according to claim 16, further comprising a therapeutically effective amount of a hormone selected from the group consisting of dehydroepiandrosterone, progesterone, pregnenolone, melatonin, and any combination thereof.

23. The transdermal pharmaceutical composition according to claim 16, further comprising a therapeutically effective amount of an amino acid selected from the group consisting of arginine, glutamine, lysine, phenylalanine, tyrosine, GABA, tryptophan, carnitine, acetyl-l-carnitine, and any combination thereof.

24. The transdermal pharmaceutical composition according to claim 16, further comprising a therapeutically effective amount of a fatty acid selected from the group consisting of a fish oil, a flax seed oil, and a combination thereof.

25. The transdermal pharmaceutical composition according to claim 16, further comprising a therapeutically effective amount of coenzyme Q10.

26. The transdermal pharmaceutical composition according to claim 16, further comprising a therapeutically effective amount of a cartilage building nutrient selected from the group consisting of glucosamine, chondroitin, MSM, and any combination thereof.

27. The transdermal pharmaceutical composition according to claim 16, further comprising a therapeutically effective amount of a herb selected from the group consisting of ginkgo biloba, echinacea, 5-HTP, St. John's wort, saw palmetto, and any combination thereof.

28. The transdermal pharmaceutical composition according to claim 16, wherein said pluronic lecithin organogel comprises a mixture of a soy lecithin/isopropyl palmitate and a pluronic organogel.

29. The transdermal pharmaceutical composition according to claim 16, wherein said imbalances caused by calcium deficiency are selected from the group consisting of hypertension, high cholesterol, colon and rectal cancer, osteomalacia, rickets, osteoporosis, cardiovascular disease, preeclampsia, tooth decay, and premenstrual syndrome.

30. A method of reducing the likelihood of calcium deficiency or imbalances caused by calcium deficiency in a human comprising the steps of topically administering to said human a transdermal pharmaceutical composition comprising:
a therapeutically effective amount of a pharmaceutically acceptable salt of calcium; and
a pharmaceutically acceptable carrier comprising a pluronic lecithin organogel;
wherein a dosage of said composition is rubbed onto an outer layer of skin of said human to achieve said transdermal delivery of said therapeutically effective amount of said pharmaceutically acceptable salt of calcium.

31. The method according to claim 30, wherein said therapeutically effective amount of calcium is in the range of 10 mg to 150 mg per dosage.

32. The method according to claim 31, wherein therapeutically effective amount of calcium is 40 mg per dosage.

33. The method according to claim 30, wherein said pharmaceutically acceptable salt of calcium is calcium pantothenate.

34. The method according to claim 30, further comprising a therapeutically effective amount of a pharmaceutically acceptable salt of a mineral selected from the group consisting of magnesium, zinc, selenium, manganese, chromium, and any combination thereof.

35. The method according to claim 30, further comprising a therapeutically effective amount of a vitamin selected from the group consisting of vitamin A, vitamin D, vitamin C, vitamin E, vitamin B₆, vitamin B₁₂, vitamin B₃, vitamin B₅, vitamin B₂, vitamin B₁, folic acid, choline, lecithin, inositol, PABA, biotin, bioflavonoids, and any combination thereof.

36. The method according to claim 30, further comprising a therapeutically effective amount of a carotenoid selected from the group consisting of lycopene, lutein, and a combination thereof.

37. The method according to claim 30, further comprising a therapeutically effective amount of a hormone selected from the group consisting of dehydroepiandrosterone, progesterone, pregnenolone, melatonin, and any combination thereof.

38. The method according to claim 30, further comprising a therapeutically effective amount of an amino acid selected from the group consisting of arginine, glutamine, lysine, phenylalanine, tyrosine, GABA, tryptophan, carnitine, acetyl-l-carnitine, and any combination thereof.

39. The method according to claim 30, further comprising a therapeutically effective amount of a fatty acid selected from the group consisting of a fish oil, a flax seed oil, and a combination thereof.

40. The method according to claim 30, further comprising a therapeutically effective amount of coenzyme Q10.

41. The method according to claim 30, further comprising a therapeutically effective amount of a cartilage building nutrient selected from the group consisting of glucosamine, chondroitin, MSM, and any combination thereof.

42. The method according to claim 30, further comprising a therapeutically effective amount of a herb selected from the group consisting of ginkgo biloba, echinacea, 5 HTP, St. John's wort, saw palmetto, and any combination thereof.

43. The method according to claim 30, wherein said pluronic lecithin organogel comprises a mixture of a soy lecithin/isopropyl palmitate and a pluronic organogel.

44. The method according to claim 30, wherein said imbalances caused by calcium deficiency are selected from the group consisting of hypertension, high cholesterol, colon and rectal cancer, osteomalacia, rickets, osteoporosis, cardiovascular disease, preeclampsia, tooth decay, and premenstrual syndrome.
